(19) United States
(12) Patent Application Publication
(10) Pub. No.: US 2025/0113875 A1
(43) Pub. Date: Apr. 10, 2025

(54) SMOKING CESSATION DEVICE AND METHOD OF USE

(71) Applicant: MacKenzie Rae Matthews, Chemainus (CA)

(72) Inventor: MacKenzie Rae Matthews, Chemainus (CA)

(21) Appl. No.: 18/907,870

(22) Filed: Oct. 7, 2024

Related U.S. Application Data
(60) Provisional application No. 63/588,180, filed on Oct. 5, 2023.

(51) Int. Cl. A24F 47/00; A24F 42/20; G16H 20/13

(52) U.S. Cl. CPC A24F 47/00; A24F 42/20; G16H 20/13

(57) ABSTRACT

A smoking cessation device features a handheld elongated body one or more mouth openings at or near the one end and receivable in a mouth of the user in a gripped state of the body between lips of the user, and at a distance from the mouth opening(s), one or more air admittance openings through which ambient air is introducible into the elongated body near another end thereof. An entirely empty internal passageway fluidly communicates the air admittance opening(s) with the mouth opening(s) such that inhalation by the user with the device in the working position is operable to draw only ambient air, with zero additives, into the mouth of the user. Preferably one or more flow restrictors are provided to mimic the drag of a cigarette or pull of a vape, preferably in accompaniment with a flow adjuster for variable control over such flow restriction for personalized experience.

### CROSS-REFERENCE TO RELATED APPLICATIONS

This application claims benefit under 35 U.S.C. 119(e) of U.S. Provisional Patent Application No. 63/588,180, filed Oct. 5, 2023.

### FIELD OF THE INVENTION

The present invention relates generally to smoking cessation devices, and associated methods of use.

### BACKGROUND

Smoking cessation devices currently available to the public require continuously buying cartridges or refills that contain chemicals or additives, such as flavouring agents, nicotine, etc. Examples of such devices include vapes, or vaporless essential oil inhalers or inhalers with other additives. For example, cartridges can contain things like chemicals, a multitude of additives, synthetic fibres and oils. Inhaling anything directly into your lungs other than naturally pure ambient air may have detrimental health effects. Given this background, there is a need for smoking cessation solutions that don't come with the potential health side effects of the current solutions available in the marketplace.

### SUMMARY OF THE INVENTION

According to one aspect of the invention, there is provided a smoking cessation device configured to mimic a smoking implement, the smoking cessation device comprising:
an elongated body configured to be held in a hand of a user and having first and second ends of opposing relationship to one another in a longitudinal direction in which said elongated body is of elongated character;
at a mouth portion of the elongated body that includes the first end of the elongated body and is longitudinally distant from the second end thereof, one or more mouth openings receivable in a mouth of the user in a working position of the cessation device in which said mouth portion is gripped between lips of the user;
at an air admittance portion of the elongated body situated at a longitudinal distance from the mouth opening, one or more air admittance openings through which ambient air is introducible into the elongated body in one or more at least partially open states of said one or more air admittance openings; and
inside the elongated body, an internal passageway by which the one or more air admittance openings are fluidly intercommunicated with the mouth opening such that inhalation by the user with the device in said working position is operable to draw said ambient air into the mouth of the user.

In some embodiments, at least one of the or more mouth openings is different in at least one of either area size or shape from at least one of said at one or more air admittance openings.

In some embodiments, the one or more mouth openings is larger in area size than the one or more air admittance openings.

In some embodiments, at least one of the one or more mouth openings and the one or more air admittance openings is smaller in area than a cross-sectional area of the passageway, thereby forming a restriction to flow of said ambient air through the passageway.

In preferred embodiments, a flow adjuster operably installed on the elongated body in movable relationship to the one or more air admittance openings between a plurality of different positions of different obstructiveness to said one or more air admittance openings to adjust an overall openness thereof and thereby vary an adjustable flow-restriction imparted thereby to the ambient air inhaled through the internal passageway.

In some embodiments, said flow adjuster comprises a cap configured for fitted engagement with the elongated body at the second end thereof.

In some embodiments, at least one of the one or more air admittance openings is located in a peripheral wall of the tubular body.

In such embodiments, the flow adjuster may comprise a circumferential wall that spans circumferentially about the elongated body, and is rotatable circumferentially therearound for movement between said different positions.

In some embodiments, the flow adjuster comprises one or more flow restriction openings therein, and each of said different positions corresponds to a different degree of registration between the one or more air admittance openings and the one or more flow restriction openings.

In some embodiments with a cap for fitted engagement with the elongated body thereof, the one or more air admittance openings include an axial air admittance opening at the second end of the elongated body, and the cap has one or more flow-restriction openings therein of smaller size than said axial admission opening to restrict flow of ambient air into the passageway through said terminal admission opening.

In some embodiments with the adjuster, the second end of the elongated body is partially open, and the adjuster comprises a rotatable cap configured for fitted engagement with the second end of the elongated body and having one or more air restriction openings therein that are movable between positions of varying registration with open and closed portions said second end of the elongated body.

Given that the device is not subject to flame, since it's use does not involve burning of any inhaled consumable, the elongated body may be composed of any variety of materials of sufficient rigidity to self-maintain a statically fixed shape in conditions of normal use, non-limiting examples of which include rigid plastic, metal, ceramic, wood of any variety, and bamboo.

Optionally, the mouth portion may be characterized by a different shape profile distinguished in at least one of size or shape from the air admission portion.

Preferred embodiments include at least one screen supported in a position of screening relationship of the one or more air admittance openings to prevent admission of contaminants into the internal passageway.

According to a second aspect of the invention, there is provided a method for mimicking use of a smoking implement comprising:
in possession of a smoking cessation device according to the first aspect of the invention, and with the first end thereof received in the mouth of the user and the mouth portion of the device gripped between the lips of the user in achievement of the working position of said device, inhaling only uncontaminated and unflavoured ambient air through the smoking cessation device into the mouth.

Preferably, an entirety of the internal passageway of said smoking cessation device, from the one or more air admittance openings to the one or more mouth openings, is fully void of any content other than said uncontaminated and unflavoured ambient air.

According to a third aspect of the invention, there is provided a method for mimicking use of a smoking implement comprising:
providing a smoking cessation device having one or more mouth openings, one or more air admittance openings spaced longitudinally therefrom and an entirely empty internal passageway fluidly intercommunicating said one or more mouth openings with said one or more air admittance openings; and
with the one or more mouth openings of the smoking cessation device received in a mouth of a user, inhaling only ambient air through the smoking cessation device.

The method may include manipulation of a flow adjuster of the smoking cessation device to change a degree of flow restriction imparted to the ambient air inhaled through said empty internal passageway.

The flow adjuster may comprise a removable cap, in which case manipulating said flow adjuster may comprise installing or removing said removable cap to or from the smoking cessation device to change said degree of flow restriction between a greater amount of flow restriction in presence of said removable cap and a lesser amount of flow restriction in absence of said removable cap.

In other instances, manipulating said flow adjuster may comprise adjusting a position of the adjuster relative to the one or more air admittance openings to change a relative obstructiveness of the adjuster to said one or more air admittance openings.

The adjuster may be a rotatable adjuster whose position is adjusted via rotational motion thereof.

Alternatively, the adjuster may be a slidable adjuster whose position is adjusted via sliding motion thereof.

### BRIEF DESCRIPTION OF THE DRAWINGS

The invention will now be described in conjunction with the accompanying drawings in which:

FIG. 1 schematically shows a cross-sectional view of a single-capped embodiment of a smoking cessation device according to the present invention, with a first end thereof received in a user's mouth.

FIG. 2 is a cross-sectional view along line 2-2 in FIG. 1.

FIGS. 3A and 3B show an uncapped embodiment of the smoking cessation device in perspective view from opposing ends thereof.

FIGS. 3C and 3D show two variants of the uncapped embodiment with different mouth opening geometries at the first end thereof.

FIGS. 3E to 3G show three further variants of the uncapped embodiment with different air admittance geometries at a second end thereof.

FIG. 4 schematically shows a dual-capped embodiment of the smoking cessation device characterized by a removable cap at each of the first and second ends thereof.

FIG. 5 shows different cap variations for the preceding single-capped and dual capped embodiments.

FIGS. 6A and 6B show one variant of the single-capped embodiment of the smoking cessation device.

FIGS. 7A and 7B show another variant of the single-capped embodiment of the smoking cessation device.

FIG. 8 shows yet another variant of the single-capped embodiment of the smoking cessation device.

FIG. 9 shows a variation on the variant of FIG. 3E.

FIG. 10A is an exploded view of another variant of the single-capped embodiment of the smoking cessation device, and FIG. 10B is an assembled view thereof.

FIG. 10C shows a variation on a tubular body of the device of FIGS. 10A and 10B with an alternative shape profile at the first end thereof receivable in the user's mouth.

FIG. 10D is an exploded view of a further variation of the device of FIG. 10A, in which both the tubular body and cap of the device are both fitted with respective screens, and the tubular body is characterized another alternative shape profile at the first end thereof.

FIGS. 11A through 11C shows another variant of the uncapped embodiment from FIGS. 3A and 3B, in which air admittance takes places radially through a peripheral wall of the device instead of axially through the second end thereof, which is a closed end in this variant.

FIGS. 12A and 12B are exploded perspective views of yet another variant of the single capped embodiment, whose cap is an internally fitting type like that of FIG. 8, but features a self-locking mechanism and is shown in two different rotational positions revealing different rows of flow-restriction openings.

FIG. 12C is a cross-sectional view of the same variant as FIGS. 12A, 12B, but in an assembled state and cross-sectioned at the plane denoted by section lines C-C to illustrate a detent locking function of the self-locking cap.

In the drawings like characters of reference indicate corresponding parts in the different figures.

### DETAILED DESCRIPTION

The accompanying figures show a smoking cessation device generally indicated at 10. The smoking cessation device 10 is configured to mimic a smoking implement, and is particularly suited for use by a smoker, a vape user, or a user of other smokable/inhalable products that is seeking to quit, or at least cut back, on use such products, which is known to detrimentally involve the inhalation of chemicals, additives and/or smoke into the lungs, as already outlined in the preceding background.

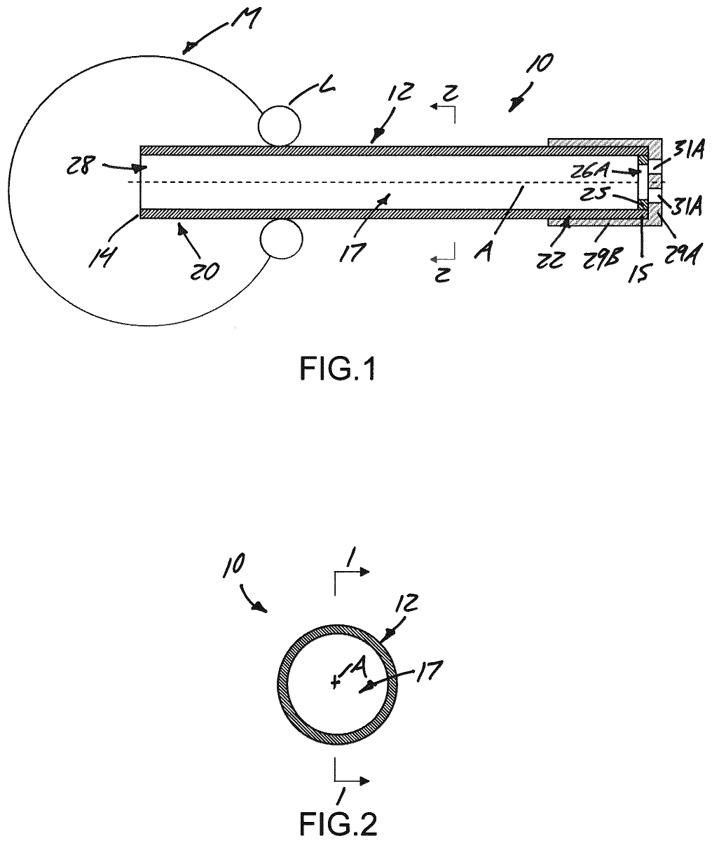

Generally speaking, the smoking cessation device 10 comprises an elongated body 12 that is typically tubular, or at least partially hollow if not fully tubular from end to end, and is suitably dimensioned and shaped, or otherwise configured, for handheld use, being easily and comfortable holdable in one hand of a user. The elongated body 12 has first and second ends 14, 15 that reside oppositely of one another in a longitudinal or axial direction denoted by a central longitudinal axis A shown in FIG. 1. It is in this longitudinal direction that the elongated body 12 of elongated character, being notably longer in this longitudinal direction that it is wide in diameter or width measured orthogonally of the axis A. In the embodiments of FIGS. 1 through 9, the elongated body 12 is tubular in shape and open at both of its first and second ends 14, 15 to permit entry and exit of ambient to and from the elongated body 12. A hollow internal passageway 17 spans longitudinally and fully from one end of the tubular body to the other in this doubly open-ended embodiment, whereby this internal passageway 17 fluidly intercommunicates the respective openings embodied at the first and second open ends 14, 15 of the tubular body 12 to enable flow of ambient air therethrough. Thus, the tubular body forms a fluidic conduit for guiding transfer of a gas between a space external to the tubular body 12 and adjacent the first end 14 thereof and another space likewise external to the tubular 12 but adjacent the second end 15 thereof.

In the illustrated embodiments, the elongated body 12 itself solely forms and delimits the passageway 17 in at least a circumferential direction around the longitudinal axis A, with a circumferential boundary wall of the passageway 17 thus being defined by an interior surface of the tubular elongated body 12, which passageway is unoccupied and uninterruptedly unobstructed between the open first and second ends 14, 15 of the elongated body 12 of the doubly open-ended embodiments to provide a continuous void therebetween. In other words, the passageway 17 is entirely empty and unobstructed throughout its full length from the opening at one end 14 to the opening at the other end 15 in such doubly open-ended embodiments, such that there is a continuous void fluidly intercommunicating these two openings with one another. The openings at the first and second open ends 14, 15 act to fluidly communicate the passageway of the tubular body with environmental spaces outside of the tubular body 12.

A mouth portion 20 of the elongated body 12 refers to partial longitudinal span thereof that includes the first end 14 and is longitudinally distant from the opposing second end 15, which mouth portion 20 is received in a mouth M of the user during use of the device, as schematically illustrated in FIG. 1. One or more mouth openings 28 are provided in the elongated body 12 at this mouth portion, so as to fluidly communicate the internal passageway with the internal oral cavity of the user's mouth. More specifically, the mouth portion 20, at a region thereof situated between the one or more mouth openings 28 and the distant second end 15 of the elongated body 12, is gripped by lips L of the user's mouth M, as schematically shown in FIG. 1, so inhalation by the user is operable to apply a suction or vacuum to the internal passageway 17 at the one or more mouth openings 28 to induce gaseous flow, in this case flow of ambient air, through the internal passageway 17 and in a longitudinally proximal direction towards the first end 14 so as to flow into the user's mouth and, in turn, into the user's respiratory system.

The illustrated embodiments are characterized a singular mouth opening 28 embodied by a fully or partially open character of the first end 14 of the elongated body 12, which mouth opening 28 thus opens axially into the interior passageway 17 of the elongated body 12, though in other embodiments, a plurality of smaller axial mouth openings may alternatively be provided in a partially closed first end of the elongated body 12. Additionally or alternatively, one or more radial mouth openings may be provided in a peripheral wall of the elongated body at the mouth portion 20 thereof, in similar fashion to radial air admittance openings 26R optionally embodied elsewhere on the elongated body 12, as described in more detail below.

An air admittance portion 22 of the elongated body 12 refers to another partial longitudinal spans thereof that includes the second end 15 and resides at a longitudinal distance from the mouth portion 20 and the one or more mouth openings 28 possessed thereby, so that during use of the device 10, this air admittance portion 22 resides externally of the user's mouth. One or more air admittance openings 26A, 26R are provided in the elongated body 12 for the purpose of admitting ambient air from the ambient environment into the internal passageway 17, from which the gaseous flow of ambient air is drawn onward into the mouth by the user through the one or more mouth openings 28, which inhalation of ambient air is performed with the device 10 in a working position in which the first end 14 is positioned in the user's mouth M with the mouth portion 20 gripped in the user's lips L. An intermediate portion of the elongated body 12 refers to a longitudinal mid-region thereof that longitudinally spans between, and longitudinally interconnects, the mouth portion 20 and the air admittance portion 22, to cooperate therewith to collectively denote the full longitudinal span of the elongated body 12.

Since the internal passageway 17 is a continuous empty void in its span between the one or more mouth openings 28 and the one or more air admittance openings 26A, 26R, the internal passageway 17 fluidly intercommunicates the oral cavity of the user's mouth with the ambient environment outside the second end 15 of the elongated body 12. The ambient air drawn into the internal passageway 17 by inhalation-created suction exerted at the mouth portion 20 by the user's mouth M is drawn through the internal passageway 17 and into the oral cavity of the user's mouth M in the same pure, uncontaminated, unflavoured state in which it entered the device 10 from the ambient environment, given the completely void status of the internal passageway 17, free any filters, additives, or removable inserts, cartridges or other carriers/refills for hosting such additives. In the illustrated embodiments, the sole openings to and from the internal passages 17 are the one or more mouth openings 28 and the one or more air admittance openings 26A, 26R, with no insertion/removal slot for receipt of inserts, cartridges, carriers, refills, etc.

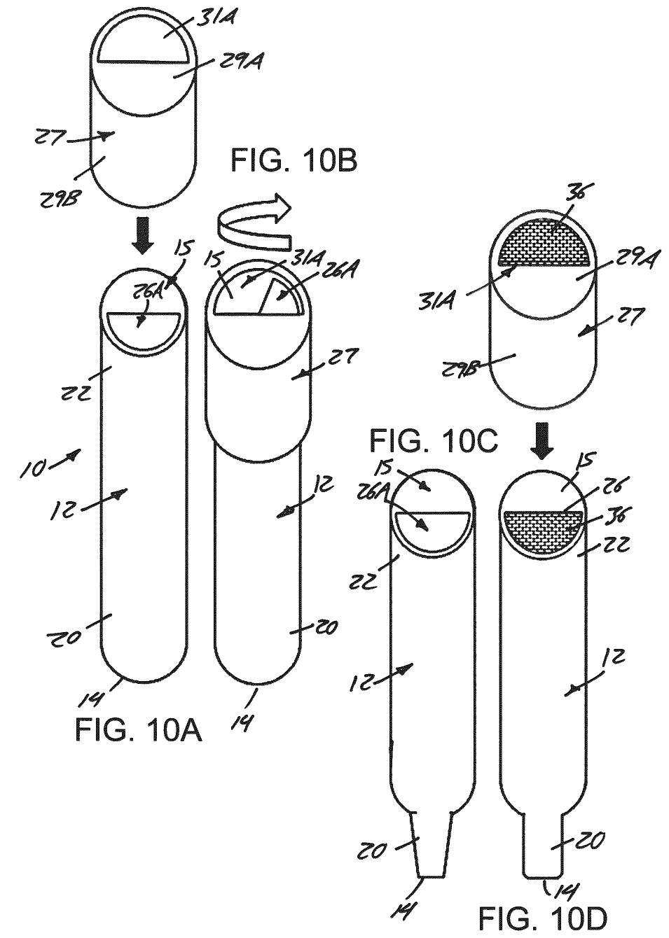

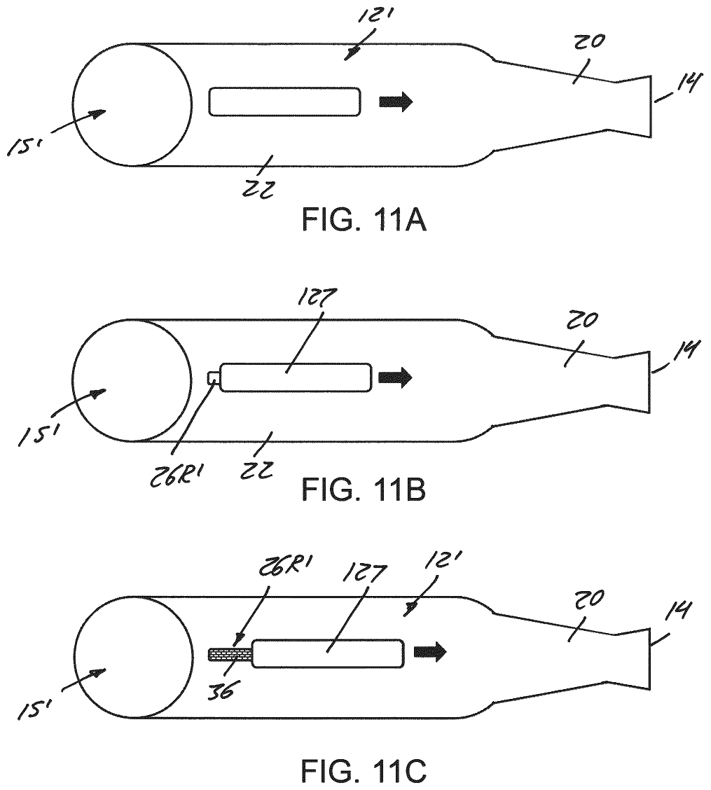

In the illustrated embodiments, it is the elongated body 12 in particular that provides a resemblance to real smoking implements, which are generally cylindrical in shape, specifically of round cylindrical character having a circular cross-section in planes normal to the longitudinal axis A, and which are linearly elongated so as to extend concentrically along an axis A. As such, the elongated body 12, in some embodiments of the present invention, is generally cylindrical in external shape throughout its entire longitudinal or axial length, and may have a substantially uniform wall thickness, such that the internal passageway 17, circumferentially bound solely by the body 12 in the illustrated examples, is also substantially cylindrical in shape and characterized by substantially uniform cross-section, in size and shape, over the full length of the body 12 between the opposite ends 14, 15 thereof. That said, the elongated body 12 need not be cylindrical over its full length, as exemplified in FIGS. 10C, 10D and 11A to 11C, where an otherwise cylindrical shape of the elongated body deviates from such cross-sectional uniformity at the mouth portion 20, where the elongated bodies in these instances instead have a reduced diameter of lesser measure that that of a cylindrical majority of the elongated body 12 that embodies the air admittance and intermediate portions thereof. This change in shape profile at the reduced-diameter mouth portion 20 may be employed to provide a distinct gripping area for the lips L of the user. The reduced mouth portion may have a cylindrical profile of uniform diameter (FIG. 10D), a tapered profile narrowing in diameter toward the first end 14 (FIG. 10C), or a variable profile, for example one of tapered and flared profile that first narrows and then widens in its approach to the first end 14 (FIGS. 11A-11C).

Since the internal passageway 17 in the illustrated embodiments is a continuous void in its span between the one or more mouth openings 28 and the one or more air admittance openings 26A, 26R, and especially when the passageway 17 is has a smooth-walled cylindrical shape, as illustrated, there is substantially minimal resistance to suction-induced airflow through the elongated body 12, which does not accurately mimic the feel or “throat draw” of taking a “drag” or “pull” of a conventional smoking or vaping implement. Thus, to better simulate the “real” feel of the smoking or vaping experience, preferred embodiments of the invention include flow restrictors to impart fluidic resistance to airflow through the passageway 17 to increase the resistance to the orally applied suction.

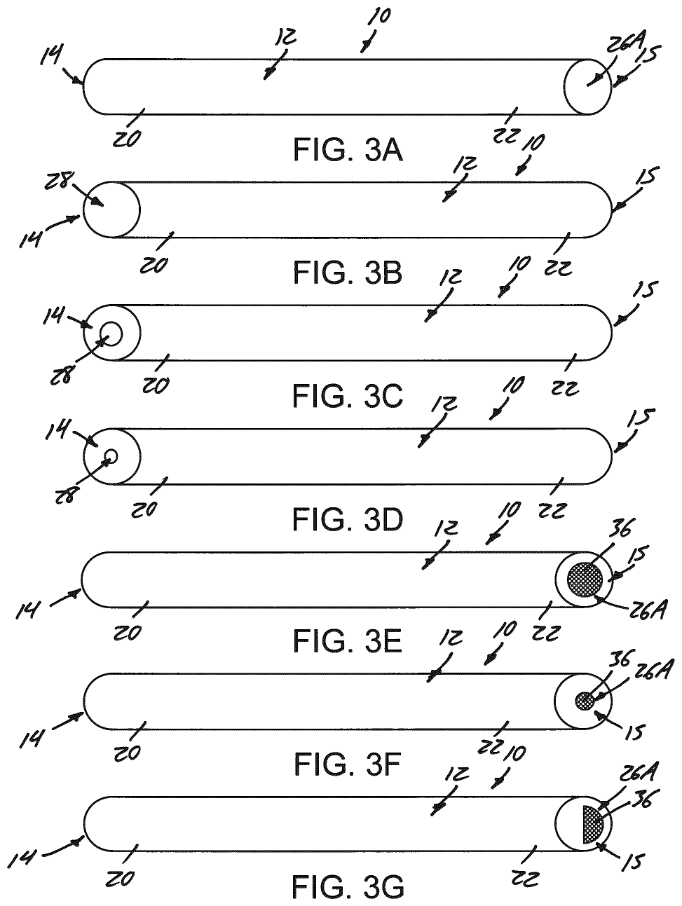

In some examples, at least one of either the mouth opening(s) 28 and/or the air admittance opening(s) 26A, 26R is characterized by a lesser cross-sectional area than that of the internal passageway, whereby the smaller opening(s) serve(s) as a flow restrictor to the flow of ambient air through the passageway 17. In the embodiment of FIG. 1, instead of leaving the second end 15 of the elongated body 12 fully open as a singular large air admittance opening (like the singular large mouth opening 28 at the first end 14), the second end 15 of the elongated body 12 is instead partially closed, for example by installation of an annular insert 25, which has a singular axial air admittance opening 26A therein, typically at a central location centered on the longitudinal axis A. The insert 25 is installed in the open second end 15 of the elongated body 12 to constrict the internal passageway 17 at that second end. It is through this axial air admittance opening 26A that ambient air is admitted to the internal passageway 17 of the elongated body 12. In contrast, the elongated body 12 in the FIG. 1 example is fully open at the first end 14 thereof, whereby this embodiment is characterized by a singular mouth opening 28 of equal cross-sectional shape and size to that of the internal passageway 17, with no flow restriction at the first end 14 of the elongated body 12. That said, as described of the second end 15, similar use of an insert to achieve a smaller opening size than the open end of the tubular elongated body 12 itself may additionally or alternative be employed at the first end 14 of the elongated body 12. FIGS. 3B and 3C show examples characterized by a singular centrally located mouth opening 28 of smaller cross-sectional area than the internal passageway 17, thus imparting airflow restriction at this first end 14 of the elongated body 12, which is achievable whether this mouth opening be provided in an integral end wall of an elongated body that is not fully open at this end, or by a separate insert 25 like that used at the opposing second end 15 in FIG. 1. FIGS. 3C and 3D illustrate how the size of the axial mouth opening 28 may be varied relative to the larger cross-sectional area of the internal passageway 17 to control the degree of airflow restriction imparted by this reduced axial mouth opening 28.

Additionally or alternatively, and with reference again to FIG. 1, the device 10 may further include a cap 27 configured to be removably and/or adjustably supported at the second end 15 of the elongated body 12. Generally speaking, the cap 27 has an end wall 29A and a cylindrical circumferential wall 29B extending axially from one side of the end wall. The cap 27 possesses one or more flow-restriction openings 31A therein that are individually or collectively characterized by a cross-sectional area that is less than that possessed by the internal passageway 17 and that possessed by the axial air admittance opening 26A at the second end 15 of the elongated body 12. These smaller flow-restriction openings 31A, in the installed position of the cap engaged on the second end 15 of the elongated body 12, are operable to impart (further) restriction to the flow of the ambient air into the passageway 17 from the ambient environment. The flow-restriction openings 31 in the cap 27 form the most-upstream openings, relative to the mouth-bound direction of ambient airflow through the device 10, for restricting the flow of ambient air into the passageway 17.

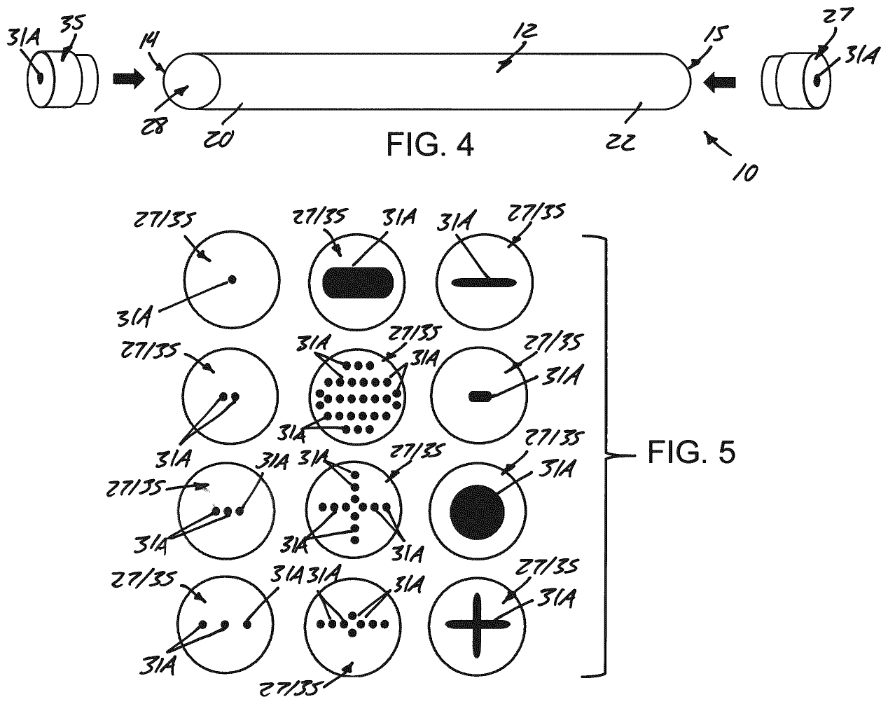

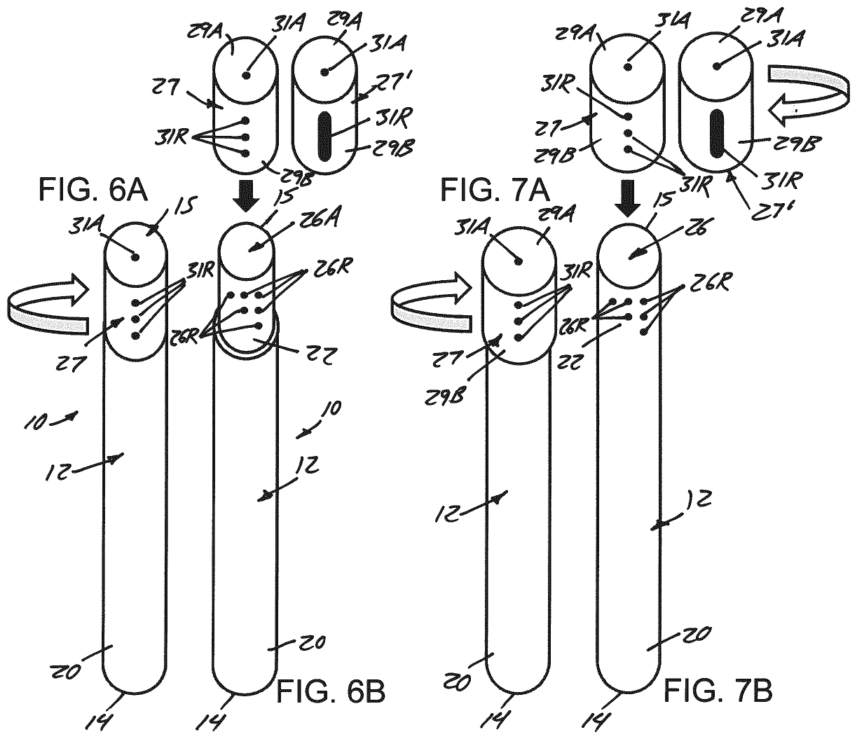

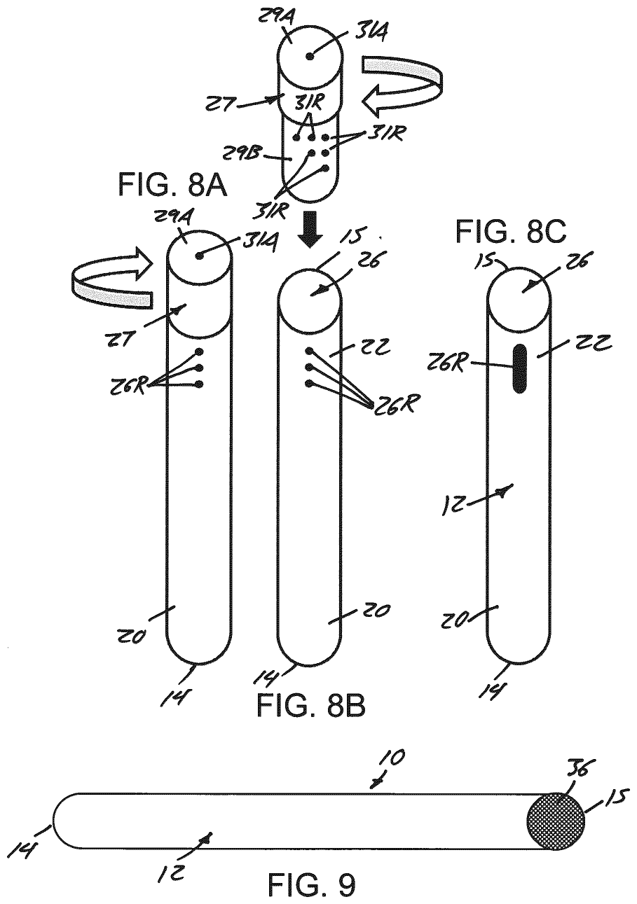

The cap 27 may be configured to be supported by the air admittance portion 22 externally or internally thereof, but in either case a slidable friction fit may be employed so that the cap is selectively removable from the elongated body 12, but self-retained on the elongated body 12 once mounted thereon. FIGS. 6 and 7 illustrate examples in which the cap fits externally over the second end 15 of the elongated body 12, in which cases the circumferential wall of the cap 27 has an internal diameter large enough to fit externally around the air admittance portion 22 of the elongated body 12. FIG. 6 illustrates an example where the air admittance portion 22 of the elongated body 12 is of reduced outer diameter relative to a remainder of the elongated body, or at least the intermediate portion thereof, and the outer diameter of the cap 27 is of generally equal outer diameter to that remainder, or intermediate portion, of the elongated body 12, so that the outer diameter of the installed cap 27 is flush with the outer diameter of said remainder, or intermediate portion, of the elongated body 12. FIGS. 4 and 8 illustrate examples in which the cap 27 instead fits internally into the second end 15 of the elongated body 12, in which case the circumferential wall of the cap 27 has an external diameter small enough for insertion into the second end 15 of the elongated body 12, which second end 15 is fully open in such examples.

In FIGS. 6 to 8 and 10, the cap 27 is rotatably supported on the air admittance portion 22 and rotatable relative thereto for selectively registering a selected one or more of the flow-restriction openings 31A, 31R with the one or more air admittance openings 26A, 26R of the elongated body 12 to enable variable flow restriction of the ambient air drawn through the internal passageway 17. That is, the resistance to suction, to provide a simulated drag on the smoking cessation device, may be adjusted through rotational manipulation of the rotatable cap 27 to adjust the degree to which the one or more air admittance openings 26A, 26R are obstructed or unobstructed by the rotationally adjustable cap 27. Examples of this type, where the cap 27 serves as a flow adjuster, are shown in FIGS. 6 to 8 and 10, in each of which the elongated body comprises one or more air admittance openings 26A, 26R and the adjuster cap 27 has one or more flow-restriction openings 31A, 31R therein, and a rotational position of the cap 27 around the longitudinal axis A enables adjustment of the degree of registration or non-registration between the overall open area denoted by the one or more air admittance openings 26A, 26R and the one or more flow-restriction openings 31A, 31R to thereby control the degree to which the air admittance openings are obstructed or unobstructed, thus dictating how much open area is available for admission of ambient air into the internal passageway 17.

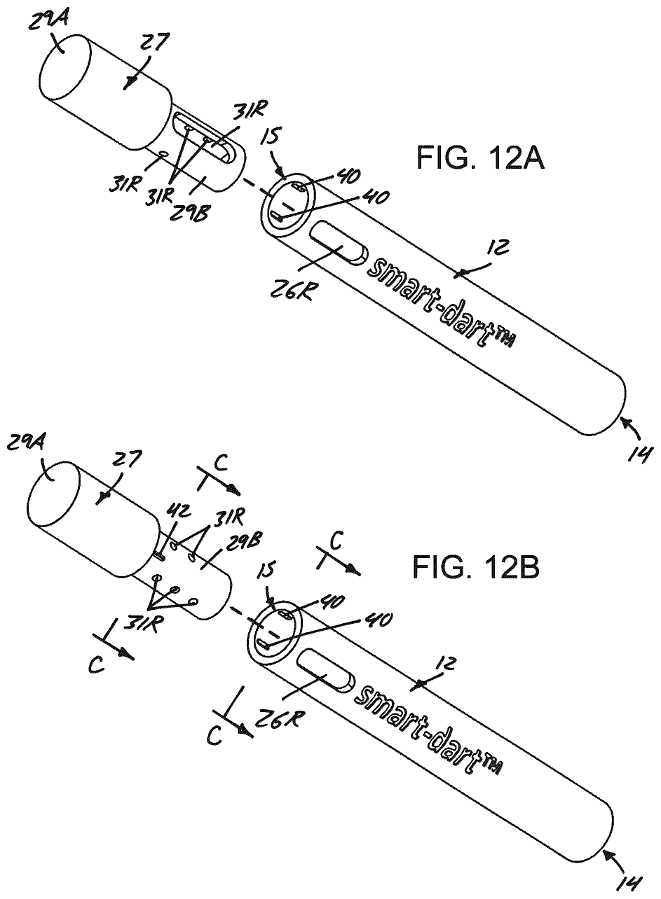

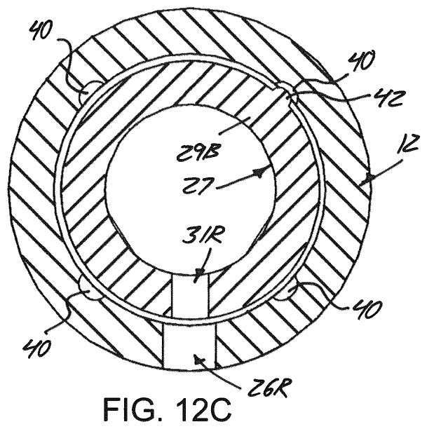

FIGS. 6 and 7 illustrated examples where multiple axial rows of radial air admittance openings 26R are provided in the peripheral wall of the elongated body 12 at the air admittance portion 22 thereof, with a different quantity of openings in each such axial row, and a singular axial row of radial flow-restriction openings 31R (left cap 27), or a singular axially elongated slot-shaped radial flow-restriction opening 31R (alternative right cap 27′), is provided on the circumferential wall 29B of the cap. Rotation of the radial flow restriction opening(s) 31R of the cap 27/27′ into alignment with a different selected one of the rows of radial air admittance openings 26R on the elongated body 12 is operable to change the overall openness of the radial air admittance openings 26R to control the amount of flow resistance imparted to the inhaled ambient airflow through the internal passageway 17. FIG. 8 illustrates how the distribution of the multi-rowed set of openings on one component and the single-row openings or single slot-shaped opening on the other component may be reversed, by providing multiple rows of radial flow-restriction openings 31R on the cap 27 to cooperate with a single row of radial air admittance openings 26R (FIGS. 8A & 8B), or single slot-shaped radial air admittance opening 26R (FIG. 8C), on the elongated body 12, to achieve the same rotatable flow adjuster functionality as FIGS. 6 and 7. In the case of the slotted cap 27′ of FIGS. 6B and 7B with the slot-shaped flow-restriction opening, the or the slotted elongated body of FIG. 8C with the slot-shaped air admittance opening, the multiple rows of holes in the other component may be accompanied by its own elongated slot of comparable size to that in the other component (as shown in FIG. 12A) to serve as a most-open row of the arrayed rows of openings, enabling flow into the passageway 17 over the full area of the slot in the other component.

As shown in each of FIGS. 6 to 8, each cap 27 may have at least one axial flow-restriction opening 31A in the end wall 29A of the cap 27 that is always in registration with the axial air admittance opening 26A at the second end 15 of the elongated body 12, whereby some degree of airflow into the internal passageway 17 is always achievable regardless of the rotational position of the cap, and the radial openings 26R, 31R are selectively registrable to varying degrees to variably supplement the baseline flow permitted by the always-registered axial openings. A rotational adjuster of same functional purpose as a rotationally adjustable cap 27 installed over the second end 15 of the elongated body 12 may alternatively be implemented as an open-ended sleeve disposed circumferentially around the air admittance portion 22, in functional equivalence to the circumferential wall 29B of the cap 27, but lacking the end wall 29B thereof.

FIG. 10 illustrates another alternative, where the elongated body is partially open at the second end 15, having an axial air admittance opening 26A of lesser size than the cross-sectional area of the internal passageway 17, for example a semi-circular area equal to about half of the internal passage's cross-sectional area, and the end wall 29A of the cap 27 likewise has an axial flow-restriction opening 31A of lesser size then the cross-sectional area of the internal passageway 17, and thus occupying only a fraction of the cap's end wall 29A, for example again being a semicircular opening equal to about half of the of the internal passage's cross-sectional area. Rotation of the cap 27 is operable to vary the openness of the air admission opening 26A by changing the degree of alignment or overlap between the air admission opening 26A and the flow-restriction opening 31A, which determines how much of the air admission opening 26A is obstructed by the cap 27.

Furthermore, the cap 27, whether serving as a rotatable adjuster or not, may be removably supported at the second end 15 such that fluidic resistance on the airflow through the internal passageway 17 is adjustable depending on whether the cap is engaged on the second end of the elongated body 12, to impart air restriction functionality to the device, or disengaged from the elongated body to permit less restrictive airflow therethrough as a result of lesser obstruction of the one or more air admittance openings 26A, 26R.

As described above, in some embodiments at least one of the one or more air admittance openings is a radial air admittance opening 26R located in a peripheral wall of the tubular body 12 to open radially into the internal passageway 17 through said peripheral wall, in which case at least one of the one or more flow-restriction openings is a radial flow-restriction opening 31R arranged in the circumferential wall 29B of the cap 27 that circumferentially encompass the air admittance portion 22 of the elongated body. The radial flow-restriction opening 31R passes radially through said circumferential wall 29B of the cap 27 to enable radial admission of the ambient air into the internal passageway 17 when the radial flow-restriction opening 31R is registered into alignment, or at least partial overlap, with one or more of the radial air admittance openings 26R in the peripheral wall of the elongated body 12. Examples of this are seen in FIGS. 6 to 8, each one of which further illustrates how such radial air admittance openings 26R and radial flow-restriction openings 31R may be accompanied by at least one axial flow-restriction opening 31A in the end wall 29A of the cap 31 and one or more axial air admittance openings 26A at the second end 15 of the elongated body 12. In other embodiments, such as the illustrated example of FIGS. 4 and 5, the cap may lack any radial flow-restriction openings 31R in the circumferential wall 29B of the cap 27, and instead comprise only one or more axial flow-restriction openings 31A. In such instance, the elongated body 12 may lack any radial air admittance openings 26R in its peripheral wall, or may include one or more such radial air admittance openings 26R, that are either obstructed by installation of the cap 27, or are instead unaffected thereby.

Typically, the flow-restriction openings 31A, 31R, such as those embodied in the cap 27, operably reside at the air admittance portion 20 of the tubular body in cooperative relationship to the air admittance opening(s) 26 so as to be located in upstream relation to the flow of ambient air through the passageway 17. However, flow-restriction openings may additionally or alternatively be provided at or adjacent the mouth portion 20 in cooperative relation to the one or more mouth openings 28, thus residing in downstream relation to airflow through the passageway 17 from the air admittance opening(s) 26 at or near the second end 15. For example, such flow-restriction opening(s) arranged at or adjacent the first end 14 of the elongated body 12 may be located in a mouth-end cap 35 engaged to the elongated body 12 at the mouth portion 20 thereof. The mouth-end cap 35 may be removably supported at the first end 14 of the elongated body 12, so that the fluidic resistance to airflow through the passageway is adjustable by selective installation and removal of the mouth-end cap 35, in the same manner described of the cap 27.

This is illustrated, for example, in FIG. 4, where the elongated body 12 is accompanied by both a mouth-end cap 35 removably engageable to the elongated body 12 at the first end 14 thereof in operative relation to the mouth opening(s), and an inlet-end cap 27 removably engageable to the elongated body at the second end 15 thereof in operative relation to the air admittance opening(s). The engagement of either cap to the elongated body 12 can be of any variety, including push-on or push-in friction fit, twist-and lock or threaded screw fit. FIG. 5 illustrates several non-limiting examples of flow-restriction hole layouts that may be used in either end cap 27, 35, including single-hole layouts and multi-hole layouts of varying hole quantity, hole size, hole shape, hole position and hole distribution.

The second end 15 of the elongated body 12 may be equipped with a screen so as to permit fluidic communication between the passageway 17 and the ambient environment but substantially prevent or resist entry of airborne debris or contaminants into the passageway 17, to avoid inhalation or swallowing thereof by the user. The intake-end cap 27, if equipped with sufficiently small openings therein, may serve as the screen, or the screen may instead take the form of a mesh screen 36 installed in operative relationship to the one or more air admittance openings 26A, 26R, whether supported directly on the elongated body 12, or instead supported on the intake-end cap 27 that is installed over the air admittance portion 22 of the elongated body 12. The fineness or porosity of the screen 36 should be one that substantially does not impede or affect airflow into the passageway. Examples of such screen implementation can be seen in FIGS. 3E to 3G, where a screen 36 installed on the elongated body 12 spans an undersized axial air admittance opening 26A at a partially open first end of the elongated body 12; FIG. 9 where a screen 36 on the elongated body 12 spans a full-sized air admittance opening at a fully open first end of the elongated body 12, and FIGS. 10C and 10D, where a first of two screens 36 spans the undersized air admittance opening at a partially closed first end of the elongated body, and a second of the two screens 36 spans the axial air restriction opening 31A in the intake-end cap 27. In this two-screen example of FIG. 10, the device has screening capability even in the event of intentionally capless use or inadvertent cap loss, yet there exists a failsafe screen redundancy in the presence of the cap in case the screen on the elongated body 12 should be damaged somehow.

FIGS. 12A through 12C (collectively, FIG. 12) illustrate optional inclusion of a snap-fit detent lock usable in implementations similar to FIG. 8 with the internally-fit rotatable cap 27, for the purpose of automatically locking the cap in any selected one of a plurality of user-selectable positions of the cap 27, each denoting registration of a respective one of the rows of radial flow-restriction openings 31R in the cap 27 with the radial air admittance opening-row or slot 26R in the elongated body 12. The illustrated example of FIG. 12 has four such self-locking positions, each corresponding a respective one of four rows of radial flow-restrictions in this example, in contrast to the three rows of the FIG. 8 example, thereby demonstrating how the number of rows, and corresponding number of preferably self-locking airflow-enabling/restricting positions, may vary. The FIG. 12 variant also illustrates how one of the rows may be constituted by a singular elongated slot-like radial flow-restriction opening.

In this four-row example with four self-locking positions, there are four catch recesses 40 provided in the interior surface of the elongated body 12, in this case at equally spaced ninety-degree intervals around the central longitudinal axis A, which is the same circumferential spacing interval found between the four rows of radial flow-restriction openings. It will be appreciated that the circumferential spacing of the rows of radial flow-restricting openings and corresponding circumferential spacing between the catch recesses 40 need not necessarily be uniform spacing of equal angular intervals around the central longitudinal axis. The cap 27, at the circumferential wall 29B thereof inserted into the elongated body 12, has a protrusive detent 42 defined externally thereon for snap fit engagement into any one of the catch recesses 40 when the cap is rotated into a positioning aligning the detent 42 with such recess 40. In the cross-sectional view of FIG. 12C, only one the four rows of flow-restriction openings is visible, as the cross-sectional plane C-C taken at the location of the protrusive detent 42 cuts through only the singular elongated slot-like radial flow-restriction opening, and not through any of the smaller radial flow-restriction openings of the other rows, though the particular location of the detent may vary.

In an alternative variant, the detent may be provided on the interior surface of the elongated body, and the catch recesses provided on the exterior of the cap's circumferential wall. Similar detent locking may be implemented in the context of an externally fitted cap 27, like those of FIGS. 6, 7 and 10, with a first one of either the catches recesses or the detent provided on an internal surface of the cap's circumferential wall 29A, and the other provided on the external surface of the elongated body 12. The FIG. 12 variant also illustrates an embodiment void of any axial flow restriction openings 31A in the cap 27, whereby all airflow into the internal passageway 17 takes place solely through the radial air admittance opening 26R of the elongated body 12 and the radial flow-restriction openings 31R of the cap 27. In variants with a self-locking cap having one or more always-open axial flow-restriction opening 31A, an additional self-locking position of the cap may be provided to lock the cap in a position closing all of the radial flow-restriction openings 31R, thereby limiting airflow into the internal passageway 17 solely to the axial flow-restriction opening 31A.

FIG. 11 illustrates an alternative embodiment of the device 10 where the elongated body 12′ is fully closed at the second end 15′ thereof, and is void of any axial air admittance openings 26A like those of the preceding embodiments, and a radial air admittance opening 26R′ is a slot of axially elongated shape in the longitudinal direction of the elongated body 12. In this embodiment, the flow adjuster, instead of being a rotatably adjustable cap 27 like that described of preceding embodiments, is a slidable adjuster 127 slidably mounted to the elongated container at a position in a manner slidable back and forth along the slot-shaped air admittance opening 26R′ to variably change how much of this slot-shaped air admittance opening 26R′ is obstructed by the slidable adjuster 127, versus how much of the slot-shaped air admittance opening 26R′ is left unobstructed and available for admission of ambient air to the internal passageway 17. As shown in FIG. 11C, the slot-shaped air admittance opening 26R′, just like the axial and radial air admittance openings 26A, 26R of any preceding embodiment, may be equipped with a mesh screen 36 for the purpose already mentioned above. Multiple slot-shaped radial air admittance openings 26R′ may be provided at spaced circumferential intervals around the elongated body, each with a respective slidable cover, or using a shared slidable sleeve that spans fully around the circumference of the elongated body 12, and is likewise axially slidable back and forth in the longitudinal direction. Each slot-shaped air admittance opening 26R′ may alternatively be replaced with a row of discrete radial air admittance openings, whose overall degree of openness is likewise adjustable by sliding of the slidable adjuster 127 back and forth along the row.

Throughout the various embodiments set forth above and illustrated in the drawings, what particularly sets this smoking cessation device, and its method of use, apart from every other device is that it does not use refills or cartridges containing any chemicals or additives. It is empty; only ambient air is present and only ambient air is inhaled by the user. In the particularly preferred embodiments incorporation a means of air restriction into the design, the smoking cessation device may alternatively be referred to as an “Air Restriction Inhaler” (ARI), setting it uniquely apart from other smoking cessation devices containing chemicals or additives. This new smoking cessation device only uses ambient air and a surprisingly satisfying “restriction” or drag sensation to optimally aide the user in their endeavour to quit smoking and/or vaping and/or other inhaled consumables.

In its broadest sense, the invention can be implemented as any device that restricts or limits the flow of ambient air when a person inhales from the device, and contains nothing that contaminates or taints the natural ambient air: no cartridges, refills, chemicals or additives. The preferred implementations with some form of flow restriction are meant to mimic the traditional throat draw and/or drag that you experience when you have a cigarette, vape, or other “smoke” type product. In even more preferable implementations, the throat draw or drag can be adjusted by the user to meet the level of enjoyment, pleasure, or satisfaction that they are accustomed to experiencing when smoking traditional products full of chemicals and/or additives. Such implementations enable altering of the amount of ambient air allowed to enter the device chamber of the device by way of a dial, rotatable cap, sliding cover, or any other method of modifying the ambient air able to be inhaled by the user through the device chamber. Any method of imparting such airflow restriction may be employed in this unique context of a smoking cessation device void of any cartridge, refill, or other means of holding chemicals, additives or any other type of substance that would be inhaled into the users lungs, regardless of what particular shape the overall device may possess, and whether it is a passive (non-electronic) device like those of illustrated embodiments, or whether it incorporates any one or more electronic components, in which case a rechargeable power source (one or more batteries) may be incorporated.

Another significant aspect is a large reduction in environmental impact. Post-consumer waste is a term used to describe what is left over when a product has been used. Cigarette butts, nicotine cartridges and refills, vape cartridges and refills, vaporless cartridges and refills, all containing chemicals and/or additives, polyester fibers, oils, etc., denote such waste, on top of which is the disposable packaging associated with those wasteful “consumables”, creating massive amounts of waste flooding into landfills every year. The device of the present invention, with no disposal-requiring consumables, eliminates the continuous and awful cycle of cartridge and refill waste. This reduction would significantly impact the environment in a positive way.

The scope of the claims should not be limited by the preferred embodiments set forth in the examples but should be given the broadest interpretation consistent with the specification as a whole.

1. A smoking cessation device configured to mimic a smoking implement, the smoking cessation device comprising:
an elongated body configured to be held in a hand of a user and having first and second ends of opposing relationship to one another in a longitudinal direction in which said elongated body is of elongated character;
at a mouth portion of the elongated body that includes the first end of the elongated body and is longitudinally distant from the second end thereof, one or more mouth openings receivable in a mouth of the user in a working position of the cessation device in which said mouth portion is gripped between lips of the user;
at an air admittance portion of the elongated body situated at a longitudinal distance from the mouth opening, one or more air admittance openings through which ambient air is introducible into the elongated body in one or more at least partially open states of said one or more air admittance openings; and
inside the elongated body, an internal passageway by which the one or more air admittance openings are fluidly intercommunicated with the mouth opening such that inhalation by the user with the device in said working position is operable to draw said ambient air into the mouth of the user.

2. The device of claim 1 wherein at least one of the or more mouth openings is different in at least one of either area size or shape from at least one of said at one or more air admittance openings.

3. The device of claim 2 wherein the one or more mouth openings is larger in area size than the one or more air admittance openings.

4. The smoking cessation device of claim 1 wherein at least one of the one or more mouth openings and the one or more air admittance openings is smaller in area than a cross-sectional area of the passageway, thereby forming a restriction to flow of said ambient air through the passageway.

5. The smoking cessation device of claim 1 further comprising a flow adjuster operably installed on the elongated body in movable relationship to the one or more air admittance openings between a plurality of different positions of different obstructiveness to said one or more air admittance openings to adjust an overall openness thereof and thereby vary an adjustable flow-restriction imparted thereby to the ambient air inhaled through the internal passageway.

6. The smoking cessation device of claim 5 wherein said flow adjuster comprises a cap configured for fitted engagement with the elongated body at the second end thereof.

7. The smoking cessation device of claim 5 wherein at least one of the one or more air admittance openings is located in a peripheral wall of the tubular body.

8. The smoking cessation device of claim 5 wherein the flow adjuster a rotatable adjuster whose position is adjusted via rotational motion thereof.

9. The smoking cessation device of claim 5 wherein the flow adjuster comprises one or more flow restriction openings therein, and each of said different positions corresponds to a different degree of registration between the one or more air admittance openings and the one or more flow restriction openings.

10. The smoking cessation device of claim 1 further comprising a cap configured for fitted engagement with the elongated body, wherein the one or more air admittance openings include an axial admission opening at the second end of the elongated body, and the cap has one or more flow-restriction openings therein of smaller size than said axial air admittance opening to restrict flow of ambient air into the passageway through said terminal admission opening.

11. The smoking cessation device of claim 5 wherein the second end of the elongated body is partially open, and the flow adjuster comprises a rotatable cap configured for fitted engagement with the second end of the elongated body and having one or more air restriction openings therein that are movable between positions of varying registration with open and closed portions said second end of the elongated body.

12. The smoking cessation device of claim 5 wherein the flow adjuster is a slidable adjuster whose position is adjusted via sliding motion thereof.

13. The smoking cessation device of claim 1 further comprising at least one screen supported in a position of screening relationship of the one or more air admittance openings to prevent admission of contaminants into the internal passageway.

14. A method for mimicking use of a smoking implement comprising:
in possession of a smoking cessation device of claim 1, with the first end thereof received in the mouth of the user and the mouth portion of the device gripped between the lips of the user in achievement of the working position of said device, inhaling only uncontaminated and unflavoured ambient air through the smoking cessation device into the mouth.

15. The method of claim 14 wherein an entirety of the internal passageway of said smoking cessation device, from the one or more air admittance openings to the one or more mouth openings, is fully void of any content other than said uncontaminated and unflavoured ambient air.

16. A method for mimicking use of a smoking implement comprising:
providing a smoking cessation device having one or more mouth openings, one or more air admittance openings spaced longitudinally therefrom and an entirely empty internal passageway fluidly intercommunicating said one or more mouth openings with said one or more air admittance openings; and
with the one or more mouth openings of the smoking cessation device received in a mouth of a user, inhaling only ambient air through the smoking cessation device.

17. The method of claim 16 comprising manipulating a flow adjuster of the smoking cessation device to change a degree of flow restriction imparted to the ambient air inhaled through said empty internal passageway.

18. The method of claim 17 wherein said flow adjuster comprises a removable cap, and manipulating said flow adjuster comprises installing or removing said removable cap to or from the smoking cessation device to change said degree of flow restriction between a greater amount of flow restriction in presence of said removable cap and a lesser amount of flow restriction in absence of said removable cap.

19. The method of claim 17 wherein manipulating said flow adjuster comprises adjusting a position of the adjuster relative to the one or more air admittance openings to change a relative obstructiveness of the adjuster to said one or more air admittance openings.

20. The method of claim 19 wherein manipulating said flow adjuster comprises rotating said flow adjuster about a longitudinal axis of the smoking cessation device.
